(19) United States
(12) Patent Application Publication
(10) Pub. No.: US 2026/0041803 A1
(43) Pub. Date: Feb. 12, 2026

(54) LABORATORY AUTOMATION SYSTEMS INSTRUMENTS AND METHODS FOR DECONTAMINATING SAME

(71) Applicant: Molecular Devices (Austria) GmbH, Puch bei Hallein (AT)

(72) Inventors: Daniele TORTORELLA, Hallein (AT); Andreas GFROERER, Bergheim (AT); Felix SPIRA, Neumarkt am Wallersee (AT)

(73) Assignee: Molecular Devices (Austria) GmbH, Puch bei Hallein (AT)

(21) Appl. No.: 19/099,552

(22) PCT Filed: Jul. 28, 2023
(86) PCT No.: PCT/IB2023/057727

§ 371 Date: Jan. 29, 2025

Related U.S. Application Data
(60) Provisional application No. 63/393,435, filed on Jul. 29, 2022.

(51) Int. Cl. A61L 2/20; A61L 2/22; A61L 9/14

(52) U.S. Cl. CPC A61L 2/208; A61L 2/22; A61L 9/14; A61L 2202/11; A61L 2202/122; A61L 2202/13; A61L 2202/14; A61L 2202/15; A61L 2209/111; A61L 2209/132; A61L 2209/135

(57) ABSTRACT

Laboratory automation systems and instruments are disclosed herein comprising components that may facilitate de-contamination of the instrument using an airborne disinfectant. Maintaining the airborne disinfectant within a predetermined range can be achieved by creating a feedback loop between generating the airborne disinfectant from a disinfectant solution. Instruments disclosed can comprise vaporization modules in communication with reservoirs of a disinfectant solution, or receive cleaning cartridges comprising the disinfectant solution, which may be energized to generate the airborne disinfectant. Decontamination methods are also disclosed herein.

### CROSS-REFERENCE TO RELATED APPLICATIONS

This application is being filed on Jul. 28, 2023, as a PCT International Patent Application that claims priority to and the benefit of U.S. Provisional Application No. 63/393,435, filed on Jul. 29, 2022, the entire contents of which are incorporated herein by reference.

### INTRODUCTION

Laboratory automation systems can comprise a plurality of instruments assembled and arranged together to work reliably in continuous and automated workflows. Conventional automated workflows can involve a biological payload (e.g., induced pluripotent stem cells (iPSCs) or mTeSR™1 media) placed in a standardized microplate format footprint and be stored within and manipulated by the laboratory automation system as needed for a given assay. In certain aspects, automated workflows can last for several months, and unwanted contaminants may enter and develop within interior chambers of the instruments that may interfere with the automated workflow and the result of the assay.

Methods to thoroughly clean and disinfect interior spaces of laboratory automation systems instruments are therefore desired to prevent such interference from both external and internal biological contaminants. It is also desired that such methods may be applied throughout the interior spaces evenly, and without damaging the equipment with highly concentrated disinfectants. Decontamination methods and instruments capable of performing decontamination cycles automatically, on demand, and without interrupting automated workflows, are also desired to reduce downtime and preserve workflows and instruments in a sanitary condition.

### SUMMARY

Methods are disclosed herein for sterilizing a microplate instrument comprising an interior chamber.

In certain aspects, methods can comprise generating an airborne disinfectant in the interior chamber from a disinfectant solution comprising H₂O₂, monitoring a cleaning parameter in the interior chamber correlated to an amount of the airborne disinfectant in the interior chamber, and maintaining the amount of the airborne disinfectant within a predetermined range for a cleaning time sufficient to sterilize the interior chamber according to a SAL-6 requirement. In other aspects, methods can comprise (i) receiving, in the interior chamber, a removable cleaning cartridge comprising a power source, an energy transmitter selected from a piezoelectric transducer and a heating element, and a disinfectant solution comprising from 0.1 wt. % to 12 wt. % H₂O₂, (ii) generating an airborne disinfectant from the disinfectant solution by energizing the energy transmitter, (iii) monitoring a cleaning parameter in the interior chamber correlated to the amount of the airborne disinfectant in the interior chamber, and maintaining the cleaning parameter within a predetermined range for a cleaning time sufficient to sterilize the interior chamber according to a SAL-6 requirement. In certain aspects, the cleaning parameter can be a relative humidity within the interior chamber, as correlated to the concentration of the airborne disinfectant. In certain aspects, the energy transmitter can be a piezoelectric transducer or a heating element.

In one aspect, generating the airborne agent can comprise receiving, in the interior chamber, a removable cleaning cartridge comprising a power source, a piezoelectric transducer, and a disinfectant solution comprising from 0.1 wt. % to 12 wt. % H₂O₂, and energizing the piezoelectric transducer. In another aspect, generating the airborne agent can comprise flowing an amount of the disinfectant solution from a disinfectant solution source into a vaporization unit and vaporizing the disinfectant solution.

Apparatus applicable to methods disclosed herein are also contemplated. Cleaning cartridges disclosed herein can comprise a cartridge body defining a disinfectant solution well and a housing disposed below the disinfectant solution well, wherein the cartridge body comprises a wall separating the disinfectant solution well from the housing, and a registration feature for positioning the cartridge body on a stage of the microplate instrument, an energy transmitter disposed proximate the wall, a chargeable power source disposed in the housing and coupled to the energy transmitter, and a controller coupled to at least one of the energy transmitter and the chargeable power source for controlling a delivery of power to the energy transmitter from the chargeable power source. In certain aspects, the energy transmitter can be selected from a piezoelectric transducer and a heating element.

In other aspects, cleaning cartridges can comprise a cartridge body defining a disinfectant solution well and a registration feature for positioning the cartridge body on a stage of a microplate instrument, and an energy transmitter disposed proximate the disinfectant solution well. In certain aspects the cleaning cartridge can be configured to receive at least one of a control signal and power when the cartridge body is positioned on a stage of the microplate instrument. In other aspects, cleaning cartridges disclosed herein can comprise a cartridge body comprising a registration feature for positioning the cartridge body on a stage of the microplate instrument and a vaporization module. In certain aspects, the vaporization module can comprise a disinfectant solution well and a heating element disposed proximate the disinfectant solution well for heating a disinfectant solution in the disinfectant solution well.

Microplate instruments are also disclosed herein and in certain aspects are adapted to operate in conjunction with cartridges and methods disclosed herein. In certain aspects, microplate instruments can comprise an interior chamber, a cleaning parameter sensor, a controller, a disinfectant solution source, and a vaporization module in communication with the interior chamber and the disinfectant solution source.

### BRIEF DESCRIPTION OF THE DRAWINGS

The following drawing figures, which form a part of this application, are illustrative of described technology and are not meant to limit the scope of the disclosure as claimed in any manner, which scope shall be based on the claims appended hereto.

FIG. 1A depicts a schematic representation of a side view of a first cleaning cartridge embodiment comprising a mechanical energy transmitter.

FIG. 1B depicts a schematic representation of a side view of a second cleaning cartridge embodiment comprising a vaporization module and thermal energy transmitter.

FIG. 1C depicts a schematic representation of a bottom view of a cleaning cartridge embodiment.

FIG. 2 depicts a schematic representation of a microplate instrument in operation with a cleaning cartridge.

FIG. 3A depicts a schematic representation of a microplate instrument comprising a vaporization module.

FIG. 3B depicts a schematic representation of a further embodiment of a microplate instrument comprising a vaporization module in communication with a disinfectant solution reservoir and a distilled water reservoir.

FIG. 4 depicts a plot showing the correlation between relative humidity and H₂O₂ concentration within the instrument during Example 1.

FIG. 5 depicts a plot showing the correlation between relative humidity and H₂O₂ concentration within the instrument during Example 2.

### DETAILED DESCRIPTION

Methods disclosed herein can sanitize or decontaminate an interior chamber of an instrument by dispersing an airborne disinfectant throughout the interior chamber of the instrument. Systems for implementing the decontamination methods within automated workflows are disclosed herein and may be adapted for immediate operation using existing microplate instrumentation without the need for (or with minor) hardware and software upgrades.

### Decontamination Methods

Methods disclosed herein can provide an efficient and controlled application of a disinfectant throughout the interior volume of an instrument to be sanitized. Generally, methods can comprise generating an airborne disinfectant from a disinfectant solution, monitoring a cleaning parameter in the interior chamber correlated to the amount of the airborne disinfectant in the interior chamber, and maintaining the cleaning parameter within a predetermined range for a cleaning time sufficient to sterilize the interior chamber according to a SAL-6 requirement.

Generally, the nature of the disinfectant is not limited to a particular class of compounds or chemicals, and can be any that are suitably dispersible within an interior chamber of an instrument such as those disclosed herein. In certain aspects, the disinfectant can be selected from the group consisting of H₂O₂, quaternary ammonium compounds, chlorine compounds, alcohols, aldehydes, iodophors, and phenolic compounds. In certain aspects, the disinfectant can be provided as a solution, for instance an aqueous H₂O₂ solution. In certain aspects, the disinfectant can comprise from about 0.1 wt. % to about 12 wt. % H₂O₂, from about 0.5 wt. % to about 8 wt. % H₂O₂, or from about 1 wt. % to about 4 wt. % H₂O₂.

Generating the airborne disinfectant in methods disclosed herein can comprise any technique that allows a sanitizing concentration of the disinfectant to be dispersed through the interior chamber to be sanitized. For instance, aerosols of a disinfectant solution may be generated by energizing a surface of the solution surface using a vibrational energy source, such as a piezoelectric transducer. Mechanical forces may also be applied to generate a spray, a mist, or a fog of the disinfectant solution, as will be understood by those of skill in the art.

The airborne disinfectant may be generated inside or outside of the interior chamber to be sanitized. For instance, the disinfectant may be vaporized in a vaporization unit positioned outside the interior chamber, prior to dispersing the disinfectant vapor within the interior chamber of the instrument. Alternatively, the disinfectant solution may be flowed from a disinfectant solution reservoir and through a spray nozzle directly within the interior chamber. In still further aspects, generating the airborne disinfectant can comprise inserting a cleaning cartridge containing a disinfectant solution into the interior chamber from a position outside the interior chamber, and energizing the disinfectant solution. Unexpectedly, cleaning cartridges capable of energizing the disinfectant solution as disclosed herein can have dimensions that are substantially similar to, identical to, or smaller than, dimensions of any standard microplate as commonly employed throughout laboratory automation systems and instruments. In this manner, methods disclosed herein can be easily incorporated within existing automated processes that transfer standardized microplates between laboratory instrumentation in an automated manner.

Receiving the cleaning cartridge as referred to herein can comprise transferring a cartridge from a microplate storage rack to a microplate analysis module, microplate reader, or incubator, and into the interior chamber of the instrument. Alternatively, the cleaning cartridge can be stored within the microplate instrument, and transferred from an inactive position to an active position as needed to conduct the disinfection cycles. In this manner, receiving the cartridge in the interior chamber can be incorporated within continuous, automated sanitizing processes. Alternatively, the cartridge may be received within the interior chamber by manually inserting the cartridge within the interior chamber. In such aspects the disinfectant solution also may be loaded manually, for instance by filling the disinfectant solution to a preferred fill height within the cartridge, and loading the cartridge into the interior chamber.

In further aspects, the cleaning cartridge can comprise a disinfectant solution prior to loading the cartridge into the interior chamber or active position. Thus, in certain aspects, methods disclosed herein can comprise receiving a cleaning cartridge comprising a power source, an energy transmitter (e.g., a piezoelectric transducer, a heating element), and a disinfectant solution comprising about 0.1 wt. % to about 12 wt. % H₂O₂ within the interior chamber of an instrument. In certain aspects, the cleaning cartridge may be configured to generate an airborne disinfectant from the disinfectant solution by powering the energy transmitter from a power source onboard the cartridge, the power source initiated by a signal received from a sensor within the interior chamber, either through wired connection with the cartridge or by a wireless receiver. In this manner, no fluid lines are necessary to interface between a disinfectant solution source and the cartridge to generate the airborne disinfectant within the interior chamber.

In other aspects, cleaning cartridges may be powered by a source native to the instrument, and disconnected from the cartridge until loaded within the interior chamber. For instance, a pin connector can be present on the bottom surface of the cartridge that mates with a complementary connector of the instrument when positioned within an active position within the interior chamber. In certain aspects, the cartridge can comprise a registration feature for aligning the cartridge in the active position and ensuring a stable connection is made. In other aspects, the data and power connections between the cartridge and the instrument can be wireless, relying on the same principle of aligning components of the cartridge and instrument e.g., by aligning a registration feature of the cartridge to the instrument.

Methods disclosed herein can comprise dispersing an airborne disinfectant throughout the interior chamber in any manner suitable to efficiently distribute the disinfectant. In certain aspects, methods can comprise generating a spray, a mist, or a fog of the disinfectant solution, however, such aspects may require relatively complex mechanical components (e.g., pumps, atomizers). Alternatively, methods can comprise generating an aerosol of the disinfectant solution, e.g., by energizing a piezoelectric transducer to produce an aerosol of the disinfectant solution. Piezoelectric transducers may be tuned to specific frequencies and power levels to generate an aerosol of the disinfectant solution with a relatively minimal power requirement, given the parameters of the disinfectant solution. Generating an aerosol as referred herein can comprise energizing a piezoelectric transducer at any appropriate frequency and power. In certain aspects, the piezoelectric transducer may simply adopt a frequency of an alternating current power source, such as an alternating current source having a frequency of 60 Hz. Alternatively, the piezoelectric transducer may be tuned to generate a specific frequency when energized and controlled between pulsing of an on/off state. In certain aspects, the frequency can be about 0.1 kHz to about 100 kHz, about 20 kHz to about 100 kHz, or about 50 kHz to about 100 kHz. In other aspects, the energizing the disinfectant solution can be conducted using a variable frequency between values of any range described above.

The disinfectant solution may in certain aspects be selected based on the ability to disperse the disinfectant throughout a volume, such as by creating an aerosol, vapor, or spray of the disinfectant, and retaining its disinfecting function. In certain aspects, the airborne disinfectant agent can be generated from a disinfectant solution comprising 0.1 wt. % to 12 wt. % H₂O₂ as an aerosol, by energizing the disinfectant solution using a piezoelectric transducer as described above. In other aspects, generating the airborne disinfectant can comprise vaporization of the disinfectant solution comprising the disinfectant, and so the disinfectant solution also may be selected based on the ability to generate a vapor of the disinfectant. Vaporization can be achieved by introducing the disinfectant solution into a vaporization module, and heating the disinfectant solution until the vapor is produced.

Conventional instruments are able to adjust the relative humidity within an interior chamber of an instrument by introducing water into a vaporization module and heating the water, thereby generating water vapor that may diffuse into and throughout the interior chamber. The relative humidity can be monitored by sensors within the interior chamber, and thus creates a feedback loop to the heating element of the vaporization module via a controller to maintain the relative humidity within a predetermined range. Conventional instruments are typically able to adjust the operating temperature of the interior chamber above room temperature, for example in a range from about 20° C. to 100° C., or from about 25° C. to about 70° C.

Surprisingly, methods disclosed herein can maintain a vapor concentration within the interior chamber by creating a similar feedback loop based on a correlation of the relative humidity increase during vaporization to the amount of airborne disinfectant as a vapor. This is particularly surprising given that the relationship between the relative humidity and the airborne disinfectant is expected to vary throughout vaporization due to differing heat of vaporization temperatures between the solvent and the disinfectant within the solution, resulting in differing rates of vaporization. Differing rates of vaporization ultimately can lead increases in the concentration of the disinfectant solution that potentially distort the correlation with relative humidity.

However, methods disclosed herein may overcome this previously unrecognized problem by repeatedly vaporizing only a portion of a total amount of the disinfectant solution to be vaporized within a vaporization module. In this manner, the ratio of disinfectant to solvent in the disinfectant solution remains relatively constant throughout the sterilization procedure, as does the correlation between the airborne disinfectant (e.g., H₂O₂ vapor) and the airborne solvent (e.g., relative humidity). Additionally, the increased number of vaporization cycles also can preserve the correlation to the airborne disinfectant where decomposition rates of the airborne disinfectant and the airborne solvent differ. For instance, where the amount of H₂O₂ falls more quickly than the relative humidity following termination of vaporization cycle, application of a larger number of small volume vaporizations can be used to preserve the correlation to the airborne H₂O₂ concentration.

In certain aspects, the portion of the disinfectant solution vaporized in any single vaporization can be about 1 mL to about 25 mL, about 2 mL to about 10 mL, or about 5 mL to about 10 mL, whereas the total amount of the disinfectant solution to be vaporized during can be about 5 mL to about 250 mL, about 10 mL to about 100 mL, or about 20 mL to about 50 mL, over the course of the decontamination method. Vaporization during decontamination methods disclosed herein can comprise any number of cycles, e.g., from about 2 cycles to about 250 cycles, from about 3 cycles to about 25 cycles, or from about 5 to about 10 cycles. It will be appreciated that the number of cycles relative can have an inverse relationship with the amount of the disinfectant solution flowed into the vaporization module, where increasing the amount of cycles can allow a decreased amount of disinfectant solution to be vaporized, and therefore finer control over the amount of airborne disinfectant generated. In certain aspects, a frequency of the vaporization cycle can be static or dynamic throughout the process, and in certain aspects, can be dependent on the desired rate of change within a given point of the cleaning method.

Moreover, methods comprising repeated vaporization of small amounts of a disinfectant may improve the safety of the decontamination process. At standard conditions, pure water boils at 100° C., while a H₂O₂ aqueous solution can start boiling at a little higher temperature, depending on H₂O₂ wt. %. Thus, attempts to vaporize a relatively large amount of an aqueous H₂O₂ solution located in a sufficiently big vaporization chamber can result in a dynamic H₂O₂ wt. % concentration increase as water is preferentially evaporated from the solution. As consequence, dangerous explosive conditions may be reached in a relatively short time as H₂O₂ becomes concentrated within the vaporization module. Pulsed, sequential, or serial vaporization cycles as disclosed herein can ensure that the disinfectant solution is completely vaporized, without creating the possibility of explosive conditions.

It is further contemplated to monitor an amount of the airborne disinfectant within the interior chamber, and maintaining the amount within a predetermined range in response to a cleaning parameter falling outside the predetermined range. In certain aspects, the amount of the airborne disinfectant may be monitored directly, e.g., by the use a sensor configured to detect the amount of the airborne disinfectant within the interior chamber. As a non-limiting example, an amount of airborne H₂O₂ present within the chamber as an aerosol or vapor may be monitored directly within the interior chamber of an instrument by using a H₂O₂ sensor placed within the interior chamber.

Alternatively, and surprisingly, monitoring the amount of the airborne disinfectant can comprise measuring a cleaning parameter correlated to the amount of the airborne disinfectant in the interior chamber. In this manner, the amount of airborne disinfectant within the interior chamber may be monitored indirectly. For example, it was unexpectedly found that the amount of H₂O₂ aerosol generated within the interior chamber from an aqueous disinfectant solution comprising 0.1 wt. % to 12 wt. % H₂O₂ has a direct, proportional, and repeatable correlation to the increase in relative humidity resulting from the aerosol generation using a piezoelectric transducer, and also resulting from the vaporization of an aqueous H₂O₂ solution. Thus, the amount of H₂O₂ within the chamber may be determined by monitoring relative humidity data generated using a relative humidity sensor and correlating the data to an amount of H₂O₂ in the chamber. It will be appreciated that other cleaning parameters may also be correlated to the amount of airborne disinfectant in a similar manner. However, the correlation between the amount of disinfectant and the relative humidity is particularly advantageous as relative humidity sensors are readily available, accurate, and often present within existing microplate instrumentation.

Thus, in certain aspects, monitoring the cleaning parameter can comprise monitoring relative humidity, and maintaining the cleaning parameter within the predetermined range can comprise maintaining the relative humidity within the interior chamber in a range from about 10% to about 90%, or within any other range disclosed herein. In certain aspects, the conditions of the interior chamber may be maintained to prevent or reduce condensation of the airborne disinfectant throughout the decontamination process. As localized droplets of a condensed disinfectant are known to be more reactive than airborne disinfectants, reducing or preventing condensation of the airborne disinfectant can significantly reduce corrosion observed within the microplate instrument and its components, e.g., within the interior chamber of the microplate instrument.

Further still, monitoring the amount of airborne disinfectant within the interior chamber can comprise calculating the cleaning parameter according to known relationships between the amount of airborne disinfectant generated and the expected decay of the airborne disinfectant. In this sense, the amount of airborne disinfectant may be deduced from cleaning parameters affecting the generation and decay of the airborne disinfectant, and in certain aspects can include temperature, pressure, time, or combinations thereof. Those of skill in the art will appreciate that monitoring steps that require computation or processing may be conducted by transmitting sensor data related to the amount of the airborne disinfectant to a processor configured to process the sensor data into the desired result. In certain aspects, monitoring the amount of the airborne disinfectant can comprise measuring the cleaning parameter using a cleaning parameter sensor, transmitting the data to a processor, and processing the cleaning parameter data to determine an amount of the airborne disinfectant. Similarly, maintaining the amount of airborne disinfectant within the interior chamber also can rely on processors to determine whether the amount of airborne disinfectant is inside or outside a predetermined range and transmitting a signal to generate additional airborne disinfectant as needed.

Predetermined ranges for the cleaning parameter can be any that are appropriate for the disinfection methods to achieve a sanitary condition within the interior chamber, e.g., according to a SAL-4 requirement, a SAL-5 requirement, or a SAL-6 requirement. As will be appreciated, the predetermined range may also be dependent on the cleaning parameter. For instance, a lower limit of the predetermined range for a given cleaning parameter can be correlated to the effectiveness of a given disinfectant, as a minimum amount of the airborne disinfectant able to generate a sanitary condition within the interior chamber. Where the cleaning parameter is relative humidity for example, a minimum relative humidity can be correlated to a minimum effective ppm H₂O₂ to achieve the desired level of disinfection. In certain aspects, the predetermined range of the cleaning parameter can be correlated to a predetermined range of the amount of the airborne disinfectant needed to achieve a desired level of disinfection. As above, a minimum limit of the predetermined range may be associated with a minimum effective amount of the disinfectant to achieve a sanitary condition, e.g., a SAL-4 requirement, a SAL-5 requirement, or a SAL-6 requirement. A maximum limit of the predetermined range may be associated with a physical limit of the airborne disinfectant or cleaning parameter (e.g., a relative humidity of 100%), or an amount of the airborne disinfectant that may be ineffective or cause damage to the instrumentation. In certain aspects, the predetermined range of the cleaning parameter can be, or can be correlated to, a concentration of airborne disinfectant (e.g., H₂O₂) in a range from about 10 ppm to about 1000 ppm, from about 50 ppm to about 500 ppm, from about 100 ppm to about 350 ppm, from about 20 ppm to about 100 ppm, or from about 15 ppm to about 50 ppm.

The cleaning time required for maintaining the cleaning parameter within the predetermined range also can be determined based on effectiveness of the procedure according to a desired sanitary condition. In certain aspects, the cleaning time can be about 30 seconds to about 4 hours, about 1 minute to about 2 hours, about 10 minutes to about 45 minutes, or about 15 minutes to about 30 minutes. As stated throughout the disclosure, conducting the methods above can produce a sanitary condition to a desired level, and in certain aspects can reduce the amount of biological contaminant within the microplate instrument by at least 80%, at least 90%, at least 95%, at least 99%, or at least 99.5%. In other aspects methods disclosed herein can reduce an amount of a biological contaminant within the microplate instrument interior chamber by a log-reduction factor of at least 4 (or 99.99%, conventionally corresponding to SAL-4), a log-reduction factor of at least 5 (or 99.999%, conventionally corresponding to SAL-5), or a log-reduction factor of at least 6 (or 99.9999%, conventionally corresponding to SAL-6). As referred herein, biological contaminants can comprise a virus, a bacteria, a spore, or portion or fragment thereof. In certain aspects, the biological contaminant can comprise a nucleic acid (e.g., DNA or RNA), a protein, or other hazardous biological substance as would be understood by those of skill in the art.

Decontamination methods disclosed herein may further comprise eliminating the disinfectant from the internal chamber of the microplate instrument, as residual airborne disinfectant or accumulated on surfaces of the instrument may interfere with subsequent operations of the instrument. In certain aspects, methods can comprise a decomposition period where the disinfectant is allowed to decompose into unreactive components. For instance, airborne H₂O₂ readily decomposes to water and oxygen gas, and therefore methods may include a decomposition period to allow this decomposition to occur. In certain aspects, the decomposition period may last from 1 minute to 10 minutes, or from 5 minutes to 30 minutes. As shown below, concentrations of less than 500 ppm H₂O₂ may decompose to undetectable amounts in less than 15 minutes, or less than 5 minutes of actively generating the airborne H₂O₂.

Certain aspects may also comprise washing surfaces of the interior chamber in order to dilute or remove residual disinfectant that may have condensed within the interior chamber during the decontamination method. In this manner, the possibility of the disinfectant interfering with subsequent operations can be eliminated by ensuring the disinfectant is not present to potentially be reintroduced as an airborne agent within the interior chamber. As contemplated herein, washing may be conducted on any surface within the microplate instrument exposed to the airborne disinfectant. In certain aspects, washing may be applied to the vaporization module and its components in direct contact with the disinfectant solution during the decontamination method. As an example, embodiments comprising a vaporization module with lines to distilled water and disinfectant solution may be susceptible to vaporizing residual disinfectant in the vaporization module where subsequent operations control relative humidity in the interior chamber by vaporizing distilled water. Washing the vaporization module can be automated, and conducted before, after, or in parallel with a decomposition of the disinfectant as described above.

Methods disclosed herein allow identification of an appropriated cleaning time and disinfectant concentration such to achieve a minimum concentration-time product (also called C*T expressed herein as ppm x minutes) depending on desired target sterilization level. Methods disclosed herein therefore can reduce the amount of biological contaminant present within the instrument beyond that of conventional sterilization techniques in a comparable cleaning time. Thus, under equal conditions, the amount of a biological contaminant remaining within the microplate instrument following methods disclosed herein can be substantially less than that of conventional sterilization techniques. In certain aspects, methods disclosed herein can comprise a concentration-time product in a range from 100 to 15,000 ppm×minutes, from 2,500 to 10,000 ppm×minutes, or from 3,500 to 5,000 ppm×minutes, and achieve a SAL-6, SAL-5, or SAL-4 requirement. In certain aspects, a SAL-6 requirement can be achieved using a concentration-time product in a range from 1,000 to 15,000 ppm×minutes, from 2,500 to 10,000 ppm×minutes, or from 3,500 to 6,000 ppm×minutes. In other aspects, a SAL-5 requirement can be achieved using a concentration-time product in a range from 10 to 5,000 ppm×minutes, from 50 to 2,500 ppm×minutes, or from 250 to 1,000 ppm×minutes.

### Laboratory Automated Systems and Instruments

Laboratory automated systems and instruments are required to perform decontamination methods as described herein. However, complete overhaul of existing systems and standards is impractical. Instrumentation is disclosed herein that may be efficiently incorporated within conventional automated systems and realize advantages of automated decontamination methods. For instance, cleaning cartridges are described herein that may facilitate decontamination methods described above by interfacing with electrical power and information processing equipment present within existing instrumentation, while having a standard microplate dimension. Instruments comprising dedicated components to facilitate automated decontamination methods are also contemplated herein.

### Cleaning Cartridges

Cleaning cartridges disclosed herein generally can be configured to contain an amount of a disinfectant solution and energize the disinfectant solution to generate an aerosol of the disinfectant solution. In certain aspects, the cleaning cartridge can comprise a cartridge body defining a disinfectant solution well and a chamber below the disinfectant solution well. In certain aspects, the cartridge body can comprise a wall separating the chamber from the disinfectant solution well. In other aspects, the cartridge body can comprise a registration feature for positioning the cartridge body on a stage of the microplate instrument.

The cartridge can further comprise an energy transmitter, a chargeable power source, and a controller coupled to at least one of the energy transmitter and chargeable power source. In certain aspects the power source, controller, and energy transmitter can be contained within the chamber of the cartridge body, having the energy transmitter positioned adjacent a wall separating the disinfectant solution well and the chamber. In this manner, the electrical components can be secured within the chamber allowing the energy transmitter to energize the disinfectant solution through the wall of the cartridge body. In other aspects, the energy transmitter may be embedded within the wall or positioned within the disinfectant solution well so as to be in direct contact with the disinfectant solution of the disinfectant solution well. In such aspects, the cartridge body can comprise electrical connections spanning the chamber and walls to allow communication between the controller, the power source, the registration feature, energy transmitter, or any combination thereof.

As referred to herein, energy transmitter will be understood to encompass devices able to receive a control signal and generate and transmit a physical effect to the disinfectant solution, either directly or indirectly. In certain aspects, the energy transmitter can be a mechanical transmitter, introducing mechanical energy into the disinfectant solution in the form of infrasonic, sonic, or ultrasonic vibrations, such as may be applied to generate an aerosol of the disinfectant solution. Thus, in certain aspects the energy transmitter may be a piezoelectric transducer. Alternatively, the energy transmitter can be a thermal transmitter, introducing thermal energy into the disinfectant solution, such as may be applied to the vaporization of the disinfectant solution. In other aspects, the energy transmitter may be a heating element.

Cleaning cartridges as disclosed herein can be configured to operate within existing microplate instruments and therefore may have an outer dimension (e.g., a length, width, height, three-dimensional shape, or two-dimensional footprint) that are substantially similar to a standard microplate dimension of any commonly employed well plate dimensions. For instance, a standard well plate can comprise 24, 96, 384, or 1536 wells, and any of those arrangements may have a standardized dimensions including a height of 14.35 mm, a length of 127.76 mm, and a width of 85.48 mm. Cleaning cartridges disclosed herein also may have standard dimensions or similar in order to universally be accepted within existing microplate instruments and automated systems.

In certain aspects, cleaning cartridges disclosed herein also can comprise at least one dimension outside the standard dimensions described above. For instance, in certain aspects a height of the cleaning cartridge can be increased relative to the standard dimensions, so as to provide additional space for electrical components within the cartridge without modifying the two dimensional footprint of the cartridge. In certain aspects the cleaning cartridge can have a height of about 5 mm to about 100 mm, about 5 mm to about 50 mm, or about 5 mm to about 25 mm. Alternatively or additionally, the footprint (e.g., length, width, or both) of the cleaning cartridge may deviate from the standard dimensions above in certain aspects.

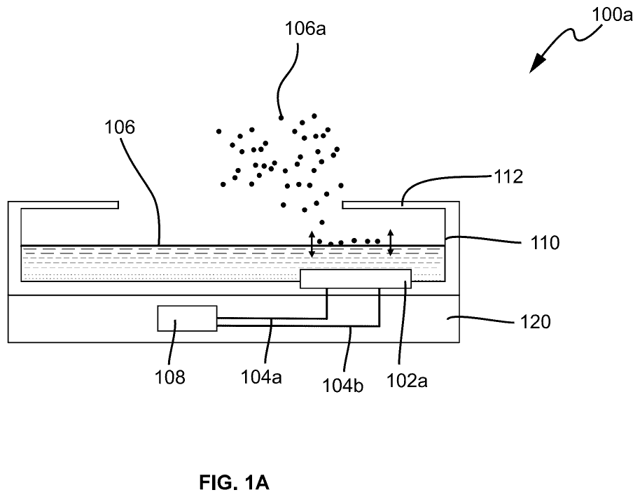

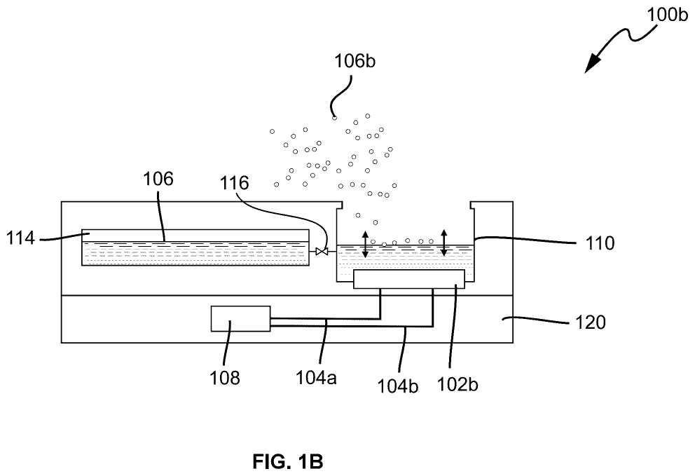

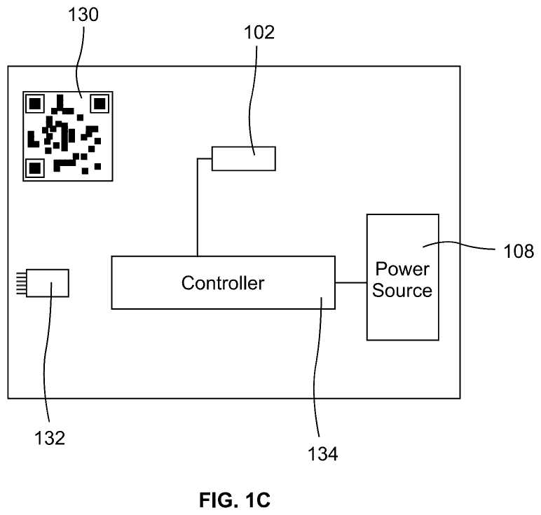

The disinfectant solution well may have dimensions similar to the standard microplate dimensions described above, with consideration the thickness of the cleaning cartridge body and wall. Thus, as pictured in FIG. 1A, the cleaning cartridge can be constructed as a ‘single-well’ plate, where the dimension of the disinfectant solution well formed by the cartridge body closely matches the footprint of the cleaning cartridge. The disinfectant solution well may also have a height that varies as described above, for instance to receive a disinfectant solution at a maximum fill height of about 5 mm to about 25 mm, or about 5 mm to about 10 mm. As shown in FIG. 1A, a distance d between the piezoelectric transducer 102 and a fill height of the disinfectant solution well can be tuned to the frequency of the piezoelectric transducer to efficiently generate an aerosol of the disinfectant solution. In certain aspects, the distance between the piezoelectric transducer and a fill height of the disinfectant solution well can be about 5 mm to about 25 mm, or about 5 mm to about 10 mm.

An amount of power consumed by the piezoelectric transducer during the cleaning cycle can be reduced by its positioning within the cleaning cartridge, among other improvements described herein. The reduction in power consumption can allow certain cartridges to be powered by a chargeable battery source throughout the duration of a cleaning cycle without needed to be recharged. Thus, in aspects wherein the chargeable power source within the cartridge is a battery sufficient to power the energy transmitter throughout the duration of a cleaning cycle, the cartridge can be employed without electrical connection to the microplate instrument, and operate independently thereof. In certain aspects, the chargeable power source can comprise a lithium ion battery. In certain aspects, the chargeable power source can have a capacity of about 0.1 to about 100 Ah, about 1 to about 20 Ah, or about 5 to about 10 Ah.

Alternatively, the power source can be a wired or wireless power receiver configured to receive power from a wireless power transmitter. Where a wired power receiver is employed, the wired power receiver may be electrical connections positioned to contact with complementary connections of the microplate instrument connected to a wired power source. In this manner, positioning the cartridge within the microplate reader brings the connections of the cartridge and the microplate instrument into physical and electrical contact, thereby providing power to the cartridge. The cartridge may also receive a control signal via similar wired connections.

In other aspects, a wireless power transmitter may be secured within a microplate reader and positioned such that when accepting the cartridge as disclosed herein, the wireless power receiver is positioned to receive power from the wireless power transmitter without a mechanical connection between the receiver and transmitter, thereby wirelessly transmitting power from the microplate instrument to the cartridge components.

In aspects comprising a piezoelectric transducer, the piezoelectric transducer can be any that apply an appropriate and sufficient vibrational force to the disinfectant solution so as to generate an aerosol. In certain aspects, the piezoelectric transducer can apply a static or dynamic frequency of about 100 Hz to about 100 kHz, about 3 kHz to about 80 kHz, or about 20 kHz to about 50 kHz. The piezoelectric transducer may also be positioned relative to cartridge components other than the fill height of the disinfectant solution to improve efficiency or durability of the cartridge. In certain aspects, the piezoelectric transducer can be positioned beneath the disinfectant solution well, and within the housing. In such aspects, the piezoelectric transducer may be positioned adjacent to the cartridge body wall to transfer of the vibrational energy to the disinfectant solution within the disinfectant solution well. In certain aspects, the cartridge body can comprise alternative materials (e.g., metal inserts) that may improve the transfer of energy into the disinfectant solution beyond the plastics typically employed in the construction of standard microplates. Alternatively, the cartridge body may be entirely constructed of a metal or a plastic, as a unitary construction.

Aspects comprising a heating element also may comprise any appropriate heating element to apply a sufficient thermal energy to generate a vapor of the disinfectant solution. Heating required to vaporize the solution may be dependent on the amount of the solution within the vaporization well or module to be vaporized. Similarly to the piezoelectric transducer, the heating element can be positioned in direct contact with the disinfectant solution or adjacent a wall of the disinfectant solution well capable of efficiently transferring heat from the heating element to the solution. In certain aspects, a probe may be connected to the heating element and inserted into the disinfectant solution for efficient heat transfer.

The disinfectant solution well may comprise additional structural features that provide advantages during disinfecting methods. As shown in FIG. 1A, the disinfectant solution well 110 can comprise an overhang that partially obstructs the disinfectant solution from splashing out of the cartridge when moved, for instance from one microplate instrument to another. In certain aspects, the overhang can extend from the edge of the disinfectant solution well toward the center of the disinfectant solution well about 5% to about 25% the relevant dimension of the cartridge. In certain aspects, the piezoelectric transducer may be at least partially disposed below the overhang of the disinfectant solution well without affecting the transmission of aerosol generated within the disinfectant solution well to the interior chamber of the microplate instrument. In this manner, splash protection is provided to prevent disinfectant solution from exiting the disinfectant solution well that may otherwise damage equipment.

Cleaning cartridges disclosed herein also can comprise a registration feature for positioning the cartridge body on a stage of the microplate instrument. The registration feature may generally be any that allow an automated positioning of the cartridge within the instrument, and therefore the registration feature can be structural feature of the cartridge body, an electrical connection such as data connections described above, an optical signal, a bar code, or any combinations thereof. In certain aspects, an outer dimension of the cartridge can operate as registration feature where the stage of the microplate instrument comprises a vertical retaining wall surrounding the footprint of a standard microplate dimension. Optical signals may also be employed as registration features, for instance where the microplate instrument has a camera or bar code reader to detect the presence of a cartridge or correlate a microplate to identifying information by barcode.

Similarly, where the stage of a microplate instrument comprises an electrical connection, the cartridge may comprise an electrical connection configured to mate with an electrical connection of the microplate instrument to relay information to and from the cartridge. For instance, in certain aspects the cartridge may receive signals from the microplate instrument to control a power sequence of the cartridge. Alternatively, an electrical connection between the cartridge and the microplate instrument upon seating the cartridge in the stage may provide power directly to the cartridge. In still further embodiments, cleaning cartridges can comprise an onboard power source to directly power the energy transmitter. In such embodiments, the cartridge may comprise a controller to generate and apply a power cycle to the piezo electric transducer within the chamber as described above.

The cartridge may further comprise a sensor providing input to the controller that may assist in determining an appropriate power cycle to achieve a desired concentration of the aerosol disinfectant within the microplate reader. In such aspects, the cartridge can be supplied with components necessary to carry out the disinfection methods in virtually any space, and therefore can be applied universally to microplate instruments without adaptation of expensive dedicated equipment with additional sensors and connections to power sources. Certain cartridges disclosed herein can comprise at least one of a humidity sensor, a hydrogen peroxide sensor, a temperature sensor, a disinfectant solution well liquid level sensor, and a proximity sensor. As discussed below, any sensor disclosed herein can be electrically coupled to a controller and transmit data thereto through an electrical connection between the cartridge and a microplate instrument.

Alternatively, components typically found within microplate instruments, including humidity sensors, power sources, and controllers, may be put to use in disinfection methods described herein without needing to duplicate the components within the cleaning cartridge. In other words, cleaning cartridges may make advantage of existing components within microplate instruments, as described for embodiments above comprising an electrical connection to a power source. Other aspects can be connected to a controller and any number of sensors within the microplate analysis module without need for onboard components within the cleaning cartridge. In such aspects, the energy transmitter may be cycled on and off using a sensor within the microplate instrument to detect a cleaning parameter, using a controller within the microplate instrument to determine whether the cleaning parameter is within a predetermined range, and using a power source to energize the energy transmitter on the cleaning cartridge.

Cleaning cartridges can comprise an electrical connection between the microplate instrument and the cleaning cartridge that allows operation of the cartridge to be controlled by components native to the microplate instrument. The electrical connection between the cleaning cartridge and the microplate instrument can be either a wired connection or a wireless connection. Thus, in certain aspects the cleaning cartridge can comprise a wireless power receiver coupled to the energy transmitter and positioned to receive power from a wireless power transmitter when the cartridge is positioned on a stage of a microplate instrument. Cleaning cartridge embodiments relying on an external power source can consist of a cartridge body comprising a disinfectant solution well, an energy transmitter, and an electrical connection to perform disinfection methods disclosed herein.

FIGS. 1A-1C provide schematic representations of non-limiting embodiments of cleaning cartridges and their components, as described above.

FIG. 1A depicts a cleaning cartridge 100a comprising a piezoelectric transducer 102a connected to a power source 108 by electrical leads 104 and in contact with disinfectant solution 106. In this manner, the cleaning cartridge is configured to generate an aerosol 106a from disinfectant solution 106 as described herein. Electromechanical transducer 102a may be configured to transmit vibrational energy into the disinfectant solution well without being in direct contact with the disinfectant solution. As shown, cartridge 100a comprises a cartridge body defining a disinfectant solution well 110 and a component housing 120. In certain aspects the piezoelectric transducer may be positioned within the housing 120 and proximate to a wall separating the disinfectant solution well and the housing. Data connections between the cartridge and a controller of an instrument and are also contemplated, and also may be present within the housing 120 to control piezoelectric transducer 102a or power source 108. In certain aspects, data connections may be electrical pins connected to the power leads of the piezoelectric transducer at one and positioned to contact pins of the instrument connected to a power source at the other end. Alternatively, the cartridge 100 can comprise a wireless receiver connected to an on-board power source further connected to the piezoelectric transducer 102a.

FIG. 1B is a second schematic representation of a non-limiting embodiment of a cleaning cartridge 100b comprising a vaporization module. Cartridge 100b comprises heating element 102b connected to a power source 108 by electrical leads 104 and in contact with disinfectant solution 106. Cartridge 100b also includes an optional reservoir 114 and optional valve 116 connecting the reservoir to vaporization well 114 to allow transfer of disinfectant solution therebetween where cycling the generation of the disinfectant solution is desired. In other aspects, the reservoir 114 and valve 116 may be excluded relying on the disinfectant solution well 110 to carry sufficient disinfectant solution for the decontamination method, as represented in FIG. 1A.

FIG. 1C presents a bottom schematic view of a cleaning cartridge embodiment disclosed herein (e.g., FIGS. 1A and 1B). As shown, the cartridge comprises QR code 130 a as a registration feature configured to be read by the microplate instrument and convey parameters related to the cartridge or sterilization method. FIG. 1C also shows a 6-pin data communication connection 132 as may be configured to interface with a complementary connection of the microplate instrument. The data communication connection can take any form (e.g. 4-pin, 8-pin, etc.) suitable for interfacing with the microplate instrument connection and internal components of the cartridge. For instance, FIG. 1C also shows the connector 132 in connection with a controller 134 which is further in connection with power source 108 and energy transmitter 102. In this manner control signals and power may be received to power any of these components from the microplate instrument, once a connection is made to connector 132. The connection may be facilitated by recognition of registration feature 130, or alternatively by physical features of the cartridge to position the cartridge appropriately to facilitate the connection between connector 132 and a complementary connection of the microplate instrument. While physical connections are shown, it is also contemplated that any of the connections and components described herein may communicate wirelessly through receivers, transmitters and the like, as will be understood by those of skill in the art.

### Laboratory Automated Systems Instruments

Laboratory automated systems instruments disclosed herein can comprise instruments for any purpose within conventional automated systems. In certain aspects, laboratory automated systems instruments can comprise include instruments not configured to receive a microplate. Alternatively, the instrument can be a microplate instrument selected from a microplate reader (e.g., a fluorescent imaging plate reader), a microplate assay module, a microplate storage module, a microplate incubator, a microplate shaker, or any combination thereof. In certain aspects, microplate instruments may be configured to receive a cleaning cartridge such as those described above for performing decontamination cycle, as part of an automated workflow. Microplate instruments can comprise a stage to receive the cleaning cartridge. In certain aspects, the stage can be a specifically dimensioned footprint, or alternatively, a shelf or a storage rack without particularly dimensioned projections. Thus, cleaning cartridges disclosed herein may be applied to practically any laboratory automation system instrument within which the cartridge may be received and energized.

Microplate instruments disclosed herein can further comprise accessory components to support the transfer of power and data between components of the microplate instrument and the cleaning cartridge. For instance, cleaning cartridges comprising a wireless power receiver can be configured for use in a microplate instrument comprising a wireless power transmitter positioned accordingly within the cartridge receiving stage of the instrument. Similarly, cartridges receiving signal input to an onboard controller and power source may be complementarily employed with a microplate reader comprising an appropriate sensor to generate the input data to the cartridge, for instance through a wireless or wired electrical connection.

Laboratory automated systems instruments are also disclosed herein that are configured to introduce airborne disinfectants independently from the use of a cleaning cartridge, instead relying on structures and components dedicated to the instrument itself. For example, instruments disclosed herein can heat a disinfectant solution within the interior chamber until the disinfectant solution is vaporized and dispersed throughout the internal space of the instrument. Instruments disclosed herein can comprise an interior chamber, a cleaning parameter sensor, a controller, a disinfectant solution source, and a vaporization module in communication with the interior chamber and the disinfectant solution source such that the disinfectant solution may flow into the vaporization module to be vaporized, and the resulting vapor may diffuse from the vaporization module into the interior chamber. Instruments comprising independent systems for generating airborne disinfectants also can include microplate instruments as described above, and may be capable of receiving, storing, analyzing, or conducting an assay using a microplate.

Laboratory automated systems instruments contemplated herein can be any shape or size as dictated by their conventional purpose, and similarly comprise interior chambers of any suitable shape and size. In certain aspects, the internal chamber can comprise narrow and hard to reach areas that can be difficult to clean, even in manual operations. The internal chamber can have a volume of about 0.1 L to about 100 L, about 0.5 L to about 50 L, or about 2 L to about 20 L.

In certain aspects, instruments can comprise one or more sensors configured to monitor a characteristic within the interior chamber for instance, and other components of laboratory automated systems disclosed herein. Generally, sensors can provide measurement data generated by the sensor to the controller, where it may be processed to dictate further action by the controller. For instance, where the disinfectant is H₂O₂, the control sensor can be an H₂O₂ sensor. As discussed above, it is also contemplated herein that sensors can include sensors of secondary characteristics that may be correlated to the amount of the airborne disinfectant. Surprisingly, the amount of airborne H₂O₂ can be reliably correlated to the relative humidity within the interior chamber, and therefore the control sensor can be a relative humidity sensor. It is also contemplated that the amount of airborne disinfectant within the chamber may be correlated to other detectable variables, including but not limited to pressure, temperature, absorbance, or combinations thereof.

In certain aspects, instruments can comprise a processor to operate components of the instrument, and transform data generated and transmitted between components of the instrument. In certain aspects, the processor may be described as a computer and may, for example, include a microprocessor, memory for data storage, instructions for data manipulation, one or more IO ports, a user interface, etc. The processor may be a dedicated component of the system or may, for example, be a personal computer with multi-purpose functionality. In some cases, the processor may be configured as a controller that controls, monitors, and/or coordinates operation of other system components, such as a pump, a sensor, a display, a printer, or any combination thereof, among others.

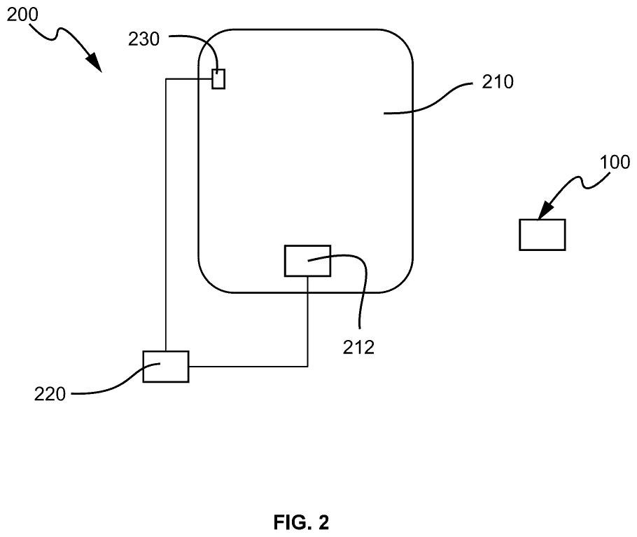

FIG. 2 is provided as a schematic representation of an aspect of a microplate instrument disclosed herein. As shown, microplate instrument 200 comprises an interior decontamination chamber 210 comprising cartridge receiver 212 capable of receiving cleaning cartridge 100 as represented in FIG. 1, and forming a data communication connection between controller 220 and the cleaning cartridge 100. In certain aspects, the data communication can be one-way communication from the instrument to the cartridge, for instance where a control signal is sent from the microplate instrument to activate a power source on the cartridge, ultimately energizing the energy transmitter. Alternatively, data communication can be one-way communication from the cartridge to the instrument, for instance where sensors related to cleaning parameters or solution levels are provided to a controller on the instrument. Further still, data communication connection can provide two-way communication between the instrument and cartridge.

As above, the data communication connection can be a physical electrical connection between the cartridge and the cartridge receiver. For instance, a registration feature may be present on the cartridge that aligns complementary connections between the instrument and the cartridge when the cartridge is properly seated on a cartridge stage within the instrument. Additionally, or alternatively, cartridges disclosed herein may receive power through such complimentary electrical connections. Thus, in certain aspects, energy transmitters of the cartridge may be controlled according by systems within the microplate instrument by providing power by closing an electrical circuit between the instrument and cartridge once the cartridge is seated within the cartridge stage (e.g., cartridge receiver 212).

Alternatively, the data communication between the instrument and cartridge may be achieved wirelessly by a wireless connection between a transmitter and receiver (or transceiver) present in either or both of the cleaning cartridge and a component of microplate instrument 200. In certain aspects, power may also be transmitted from the instrument to the receiver wirelessly, such that the cartridge may not require a dedicated power source. In such aspects, cartridges disclosed herein may comprise a wireless power receiver positioned to complement a wireless power transmitter present in the microplate instrument when the cartridge is seated within the cartridge stage (e.g., cartridge receiver 212).

As described above related to decontamination methods, cleaning parameter sensor 230 may be placed at any suitable position within the interior chamber 210 for monitoring the amount of airborne disinfectant within the chamber (or other cleaning parameter) and providing monitoring data to controller 220.

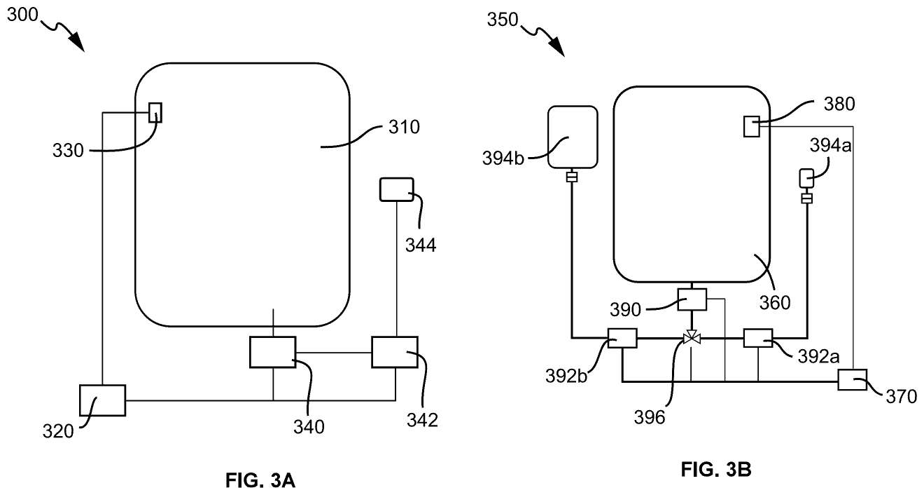

FIGS. 3A and 3B provide schematic representations of laboratory automated systems instruments configured to introduce airborne disinfectants independently from the use of a cleaning cartridge. As for the schematic representation of FIG. 2, FIG. 3A also depicts a microplate instrument 300 comprising an interior decontamination chamber 310, a controller 320, and a cleaning parameter sensor 330 configured in a similar arrangement. Instrument 300 may further comprise a cartridge receiver in certain aspects as an optional component, though not shown in FIG. 3A. As shown, instrument 300 comprises a vaporization module 340 in data communication with the controller, and in fluid communication with disinfectant solution reservoir 344 via peristaltic pump 342. In this arrangement, the controller 320 may receive data from the cleaning parameter sensor 330, and operate the peristaltic pump 342 to flow disinfectant solution from reservoir 344 to the vaporization module 340. Controller 320 may also operate a heating element (not shown) within the vaporization module 340 to vaporize the disinfectant solution, which may then diffuse into the interior chamber 310 of the instrument.

FIG. 3B also represents a laboratory instrument 350 comprising an interior decontamination chamber 360 and a vaporization module 390 connected thereto. However, vaporization module 390 is in fluid communication with both a disinfectant solution reservoir 394a and a distilled water reservoir 394b. The vaporization module 390 also is in data communication with a controller 370, as in conventional laboratory instruments where relative humidity is monitored and controlled. Reservoirs 394a, 394b each are in fluid communication with a second peristaltic pump (392a, 392b) and a selective flow unit (e.g., three-way valve 396) connecting fluid lines from each of the reservoirs to the vaporization module. Controller 370 remains in data communication with each pump 392a, b and three-way valve 396. Instrument 350 also comprises relative humidity sensor 380. In this manner, the controller 370 is configured to operate the appropriate pump 392a, b and flow water into the vaporization module from reservoir 394a to increase the relative humidity following vaporization, or alternatively, flow the disinfectant solution into the vaporization module from reservoir 394b to increase the concentration of airborne disinfectant within the solution.

In further aspects, the controller also can rely on input from the relative humidity sensor without the need for a separate or additional disinfectant sensor installed within the interior chamber. Rather the controller can rely on a correlation established between the relative humidity and the amount of airborne disinfect to modulate the amount of airborne disinfectant with accuracy and precision. Thus, as shown in FIG. 3B, microplate instruments contemplated herein can monitor both the relative humidity and amount of airborne disinfectant within the interior chamber using a single sensor.

As will be understood, microplate instruments contemplated herein are not limited to the components described above and depicted in FIGS. 2 and 3A-3B, and can comprise any amount of additional or alternative components that may be beneficial for a specific implementation. For instance, microplate instruments can comprise a stage configured to receive a standard dimensioned microplate and/or cleaning cartridge as described above. In other aspects, the instrument can be a fluorescence plate reader and comprise lamps and optical components such as monochromators, detectors fiber optic cables, and lenses.

It is to be understood that this disclosure is not limited to the particular structures, process steps, or materials disclosed herein, but is extended to equivalents thereof as would be recognized by those ordinarily skilled in the relevant arts. It should also be understood that terminology employed herein is used for the purpose of describing particular examples only and is not intended to be limiting. It must be noted that, as used in this specification, the singular forms “a,” “an,” and “the” include plural referents unless the context clearly dictates otherwise.

It will be clear that the systems and methods described herein are well adapted to attain the ends and advantages mentioned as well as those inherent therein. Those skilled in the art will recognize that the methods and systems within this specification may be implemented in many manners and as such is not to be limited by the foregoing exemplified embodiments and examples. In this regard, any number of the features of the different examples described herein may be combined into one single example and alternate examples having fewer than or more than all of the features herein described are possible.

While various examples have been described for purposes of this disclosure, various changes and modifications may be made which are well within the scope contemplated by the present disclosure. Numerous other changes may be made which will readily suggest themselves to those skilled in the art and which are encompassed in the spirit of the disclosure.

### EXAMPLES

### Example 1. Decontamination of Internal Chamber of a Liconic STX-44 Incubator to SAL-6 Requirement

Biological indicator stripes (BI) were obtained commercially from Mesa Laboratories (APEX® Log-456, 3-in-1 stripe, BI 12980). The initial population of each APEX® 12980 BI stripe is certified by supplier to be respectively not less than 10⁴, 10⁵ and 10⁶ Geobacillus stearothermophylus spores, respectively for each of the three different concentrations (log 4, log 5, and log 6). Two APEX® 12980 BI stripes were placed inside the interior chamber of a Liconic STX-44 incubator in positions that were obstructed by internal components, and therefore inaccessible to standard wiping sterilization processes. A third APEX® B1 stripe was held outside the Liconic STX-44 as a positive control. The Liconic STX-44 was equipped with a standard vaporizer containing 10 mL of an 8 wt. % aqueous H₂O₂ solution.

Decontamination of the internal chamber of the incubator was conducted according to the following automated protocol. The interior chamber was heated from ambient temperature to 50° C. over roughly 50 minutes, and maintained at 50° C. throughout the decontamination. Once at 50° C., airborne H₂O₂ was generated by repeatedly vaporizing 1-2 mL of an 8 wt. % aqueous H₂O₂ solution, until the relative humidity (RH %) exceeded 80 RH %, over approximately 15 minutes. The RH % was maintained at 80% with vaporization cycles as needed for another 15 minutes. The interior chamber was then allowed to cool to ambient temperature over 60 minutes to allow the airborne H₂O₂ to degrade into H₂O and O₂ gas. Distilled water was flushed into the vaporization module to remove residual H₂O₂ disinfectant solution.

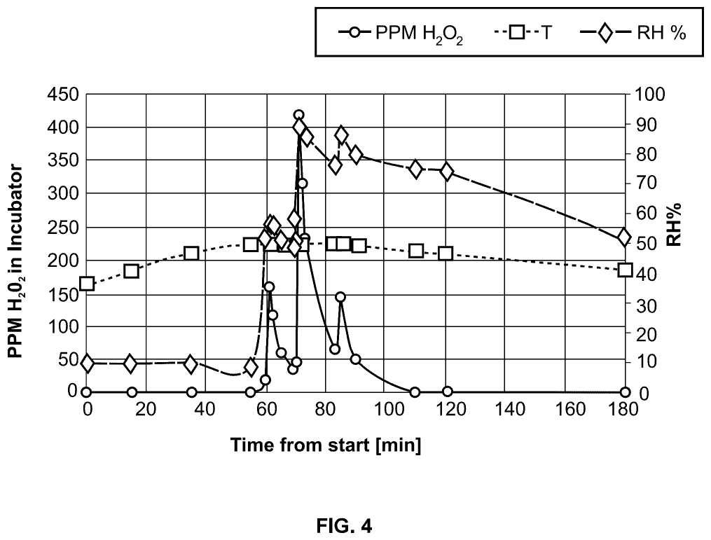

Temperature, relative humidity, and concentration of airborne H₂O₂ were monitored using a Vaisala PEROXCAP® HPP270 probe placed within the internal chamber of the incubator. Values of the temperature, relative humidity, and airborne H₂O₂ were reported throughout the decontamination process, as shown in FIG. 4. Surprisingly, a direct and proportional correlation between the relative humidity and the concentration of airborne H₂O₂ was observed throughout the decontamination process. The amount of airborne H₂O₂ was directly controlled by cycling vaporization of an H₂O₂ solution based on the amount of relative humidity measured within the interior chamber.

Three pulsed cycles were conducted, as reflected by the increase of relative humidity and H₂O₂ reflected in FIG. 4. A first vaporization cycle increased the relative humidity to about 55% and the amount of airborne H₂O₂ to reach 161 ppm. After 10 min, a second vaporization cycle increased the relative humidity to 90% and the H₂O₂ concentration to 430 ppm. A third vaporization cycle was conducted after the relative humidity dropped to 75%, which increased the airborne H₂O₂ concentration from about 60 ppm to 148 ppm. The decontamination method of this example maintained an airborne H₂O₂ concentration about 50 ppm or greater for 30 minutes, resulting in an H₂O₂ concentration-time product (C*T) of about 4,500 ppm×min. After the third vaporization cycle, the incubator was allowed to cool to room temperature over 90 minutes. The airborne H₂O₂ concentration decomposed essentially entirely within the first 30 minutes of the cool down period.

After the decontamination process, the two APEX® stripes were removed from the interior chamber and cultivated according to the manufacturer recommended protocols using Releasat Purple® cultivation media (55° C., 7 days). The control stripe was also cultivated.

After cultivating each sample according to manufacturer specifications, the media in which the positive control was cultivated changed color from purple to yellow, indicating the presence of Geobacillus stearothermophylus, as expected. In contrast, no color change was observed in the media in which samples subjected to decontamination procedure were cultivated, indicating that Geobacillus stearothermophylus was not present, and that the samples had been efficiently decontaminated. Thus, the disclosed decontamination method was able to decontaminate Geobacillus stearothermophylus spores initially present in the interior chamber to at least a SAL-6 level.

### Example 2. Parameter Optimization for Decontamination to a SAL-5 Requirement

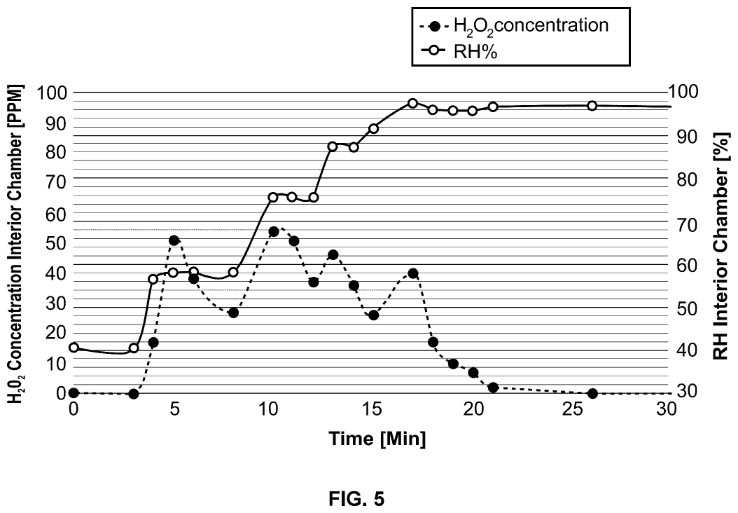

The decontamination method of Example 2 was performed according to the procedure of Example 1, except that a lesser concentrated disinfectant solution of 3 wt. % H₂O₂ was used, the cleaning parameter employed to terminate the vaporization cycle (e.g., cleaning parameter) was a relative humidity of 95%, and was targeted for a period of 20 minutes. As for Example 1, relative humidity and H₂O₂ concentration were monitored throughout the cleaning period, and results are shown in FIG. 5.

As shown, the H₂O₂ concentration was held generally between 15 and 50 ppm throughout the 20 minute cleaning period, which then quickly dissipated upon completion of the final vaporization cycle. The airborne H₂O₂ concentration does not exceed 100 ppm during the cleaning procedure, and thereby limits the exposure of equipment to the disinfectant that may eventually damage or corrode surfaces with long-term, repeated use.

The APEX® stripes were removed from the incubator after the decontamination method and cultivated using Releasat Purple® cultivation media, according to the manufacturer recommended protocol. As for Example 1, the positive control changed color from purple to yellow, indicating the presence of Geobacillus stearothermophylus, as expected. However, Apex stripes representing Geobacillus stearothermophylus populations in amounts of 10⁶ also indicating the presence of Geobacillus stearothermophylus with a color change to yellow, whereas the analogous 10⁵ population stripes observed no color change. Unexpectedly, the exposure of the internal chamber to a concentration of H₂O₂ of only 15 to 50 ppm was sufficient to sterilize the incubator to a SAL-5 requirement within an automated 30-minute procedure. Accordingly, it is demonstrated that the decontamination methods disclosed herein can be tailored based on a preferred efficiency according the to the sterility requirements of a given application to maximize sterilization efficiency, and limit equipment downtime and exposure to disinfectants that may corrode or otherwise damage the equipment over long-term use.

### Examples:

Illustrative examples of the systems and methods described herein are provided below. An embodiment of the system or method described herein may include any one or more, and any combination of, the clauses described below:

Clause 1. A cleaning cartridge for a microplate instrument, the cleaning cartridge comprising: a cartridge body defining a disinfectant solution well, and a housing disposed below the disinfectant solution well, wherein the cartridge body comprises a wall separating the disinfectant solution well from the housing, and a registration feature for positioning the cartridge body on a stage of the microplate instrument; an energy transmitter selected from a piezoelectric transducer and a heating element, the energy transmitter disposed proximate the wall; a chargeable power source disposed in the housing and coupled to the energy transmitter; and a controller coupled to at least one of the energy transmitter and the chargeable power source for controlling a delivery of power to the energy transmitter from the chargeable power source.

Clause 2. The cleaning cartridge of clause 1, wherein the energy transmitter is positioned beneath the disinfectant solution well.

Clause 3. The cleaning cartridge of any of clauses 1-2, wherein the energy transmitter is positioned adjacent to the wall.

Clause 4. The cleaning cartridge of any of clauses 1-3, wherein the energy transmitter is a piezoelectric transducer and a distance between the piezoelectric transducer and a maximum fill height of the disinfectant solution well is in a range from about 5 mm to about 25 mm.

Clause 5. The cleaning cartridge of any of clauses 1-4, wherein the cartridge body comprises a unitary plastic part.

Clause 6. The cleaning cartridge of any of clauses 1-5, wherein the cartridge body comprises an overhang at least partially enclosing the disinfectant solution well, wherein the energy transmitter is a piezoelectric transducer at least partially disposed below the overhang.

Clause 7. The cleaning cartridge of clause 6, wherein the piezoelectric transducer is completely disposed below the overhang.

Clause 8. The cleaning cartridge of any of clauses 1-6, wherein the chargeable power source is a battery sufficient to power the energy transmitter throughout a duration of a cleaning cycle.

Clause 9. The cleaning cartridge of clause 8, wherein the chargeable power source comprises a lithium ion battery.

Clause 10. The cleaning cartridge of any of clauses 1-9, wherein the chargeable power source has a capacity in a range from about 1 to about 20 Ah.

Clause 11. The cleaning cartridge of any of clauses 1-10, wherein the energy transmitter is a piezoelectric transducer configured to emit energy at a frequency in a range from about 100 Hz to about 100 kHz.

Clause 12. The cleaning cartridge of any of clauses 1-11, further comprising a sensor comprising at least one of: a humidity sensor, a hydrogen peroxide sensor, a temperature sensor, a disinfectant solution well liquid level sensor, and a proximity sensor; and wherein the sensor is coupled to the controller.

Clause 13. The cleaning cartridge of any of clauses 1-12, wherein the cartridge comprises outer dimensions substantially similar to a standard microplate dimension.

Clause 14. A method for sterilizing a microplate instrument comprising an interior chamber, the method comprising: receiving, in the interior chamber, a removable cleaning cartridge comprising a power source, an energy transmitter selected from a piezoelectric transducer and a heating element, and a disinfectant solution comprising from 0.1 wt. % to 12 wt. % H₂O₂; generating an aerosol of the disinfectant solution by energizing the energy transmitter; monitoring a cleaning parameter in the interior chamber correlated to the amount of aerosol in the interior chamber; maintaining the cleaning parameter within a predetermined range for a cleaning time sufficient to sterilize the interior chamber according to a SAL-6 requirement.

Clause 15. The method of clause 14, further comprising transferring the removable cleaning cartridge from a microplate storage into the interior chamber.

Clause 16. The method of clause 15, wherein the microplate storage is within the microplate instrument.

Clause 17. The method of any of clauses 14-16, wherein the cleaning cartridge has a standard microplate dimension.

Clause 18. The method of any of clauses 14-17, wherein transferring the cleaning cartridge is conducted by an automated system.

Clause 19. The method of any of clauses 14-18, wherein the energy transmitter is a piezoelectric transducer positioned within about 5 mm to about 25 mm from an upper surface of the disinfectant solution.

Clause 20. The method of any of clauses 14-19, wherein energizing the energy transmitter comprises energizing a piezoelectric transducer at a frequency in a range from about 100 Hz to about 100 kHz.

Clause 21. The method of clause 20, wherein the frequency is variable.

Clause 22. The method of any of clauses 14-21, wherein monitoring the cleaning parameter comprises monitoring a cleaning parameter outside the disinfectant solution.

Clause 23. The method of clause 22, wherein the cleaning parameter is hydrogen peroxide concentration.

Clause 24. The method of any of clauses 22-23, wherein the cleaning parameter is relative humidity.

Clause 25. The method of any of clauses 22-24, wherein the cleaning parameter is temperature.

Clause 26. The method of any of clauses 14-25, wherein monitoring the cleaning parameter comprises monitoring a parameter within the disinfectant solution.

Clause 27. The method of any of clauses 14-26, wherein monitoring the cleaning parameter comprises monitoring a parameter using a sensor housed within the cleaning cartridge.

Clause 28. The method of any of clauses 14-27, wherein monitoring the cleaning parameter comprises monitoring relative humidity, and wherein maintaining the cleaning parameter within the predetermined range comprises maintaining the relative humidity within the interior chamber in a range from 10% to 90%.

Clause 29. The method of any of clauses 14-28, wherein the cleaning time is in a range from 10 to 45 minutes.

Clause 30. The method of any of clauses 14-29, wherein maintaining the cleaning parameter comprises adjusting an amount of power applied to the energy transmitter.

Cause 31. The method of any of clauses 14-30, wherein an amount of a biological contaminant within the microplate reader is reduced by at least 99.99%.

Clause 32. The method of any of clauses 14-31, wherein an amount of a biological contaminant remaining within the microplate reader is less than that of conventional sterilization techniques.

Clause 33. A cleaning cartridge for a microplate instrument, the cleaning cartridge comprising: a cartridge body defining a disinfectant solution well, wherein the cartridge body comprises a registration feature for positioning the cartridge body on a stage of the microplate instrument; an energy transmitter disposed proximate the disinfectant solution well; wherein the cleaning cartridge is configured to receive at least one of a control signal and power when the cartridge body is positioned on a stage of the microplate instrument.

Clause 34. A cleaning cartridge for a microplate instrument, the cleaning cartridge comprising: a cartridge body comprising a registration feature for positioning the cartridge body on a stage of the microplate instrument; a vaporization module comprising: a disinfectant solution well; and a heating element disposed proximate the disinfectant solution well for heating a disinfectant solution in the disinfectant solution well.

Clause 35. The cleaning cartridge of clause 34, further comprising a power source coupled to the heating element.

Clause 36. The cleaning cartridge of clause 35, further comprising a controller coupled to at least one of the heating element and the power source for controlling a delivery of power to the heating element from the power source.

Clause 37. The cleaning cartridge of any of clauses 34-36, further comprising a disinfectant solution source in fluid communication with a selective flow unit, the selective flow unit further in communication with the disinfectant solution well.

Clause 38. The cleaning cartridge of any of clauses 34-37, wherein the controller is coupled to the selective flow unit for controlling a delivery of disinfectant solution from the disinfectant solution source to the disinfectant solution well.

Clause 39. A microplate instrument comprising: an interior chamber; a cleaning parameter sensor; a controller; a disinfectant solution source; and a vaporization module in communication with the interior chamber and the disinfectant solution source.

Clause 40. The instrument of clause 39, further comprising a distilled water source in communication with the vaporization module.

Clause 41. The instrument of clause 40, wherein the distilled water source and disinfectant solution source are in fluid communication with a selective flow unit, the selective flow unit further in communication with the vaporization module.

Clause 42. The instrument of any of clauses 40-41, further comprising: a first pump associated with the distilled water source; and a second pump associated with the disinfectant solution source.

Clause 43. The instrument of any of clauses 39-42, wherein the controller is configured to: receive an output from the cleaning parameter sensor; control heating within the vaporization module; control the flow of the disinfectant solution from the disinfectant solution source to the vaporizer; or any combination thereof.

Clause 44. The instrument of any of clauses 39-43, wherein the disinfectant solution source is removable from the instrument.

Clause 45. The instrument of any of clauses 39-44, wherein the cleaning parameter sensor is a humidity sensor.

Clause 46. The instrument of any of clauses 39-45, wherein the instrument is selected from the group consisting of an incubator, a microplate reader, a microplate assay module, and a microplate storage rack.

Clause 47. The instrument of any of clauses 39-46, wherein the interior chamber has a volume in a range from 5 to 500 L.

Clause 48. The instrument of any of clauses 39-47, wherein the interior chamber comprises a cartridge housing configured to receive a microplate.

Clause 49. The instrument of any of clauses 39-48, wherein the vaporization module is outside the interior chamber.

Clause 50. The instrument of any of clauses 39-49, wherein the vaporization module has a volume of about 5 mL to about 100 mL.

Clause 51. The instrument of any of clauses 39-50, wherein the vaporization module comprises a heating element.

Clause 52. The instrument of any of clauses 39-51, wherein the disinfectant solution comprises a disinfectant solution comprising from 0.1 wt. % to 12 wt. % H₂O₂.

Clause 53. A method for sterilizing a microplate instrument comprising an interior chamber, the method comprising: generating an airborne disinfectant in the interior chamber from a disinfectant solution comprising H₂O₂; monitoring a cleaning parameter in the interior chamber correlated to an amount of the airborne disinfectant in the interior chamber; and maintaining the amount of the airborne disinfectant within a predetermined range for a cleaning time sufficient to sterilize the interior chamber according to a SAL-6 requirement.

Clause 54. The method of clause 53, wherein the airborne disinfectant is a vapor or a suspension.

Clause 55. The method of any of clauses 53-54, wherein generating the airborne agent comprises a cycle, the cycle comprising: flowing an amount of the disinfectant solution into a vaporization module; and vaporizing the amount of the disinfectant solution in the vaporization module.

Clause 56. The method of any of clauses 53-55, wherein the disinfectant solution is flowed to the vaporization module from a disinfectant solution reservoir using a peristaltic pump.

Clause 57. The method of any of clauses 53-56, wherein the cleaning parameter is a concentration of airborne H₂O₂.

Clause 58. The method of any of clauses 53-57, wherein the cleaning parameter is relative humidity.

Clause 59. The method of any of clauses 53-58, wherein the cleaning parameter is temperature.

Clause 60. The method of any of clauses 53-59, wherein monitoring the cleaning parameter comprises monitoring a parameter within the disinfectant solution.

Clause 61. The method of any of clauses 53-60, wherein: monitoring the cleaning parameter comprises monitoring relative humidity; and maintaining the cleaning parameter within the predetermined range comprises maintaining the relative humidity within the interior chamber in a range from 10% to 95%.

Clause 62. The method of any of clauses 53-61, wherein the cleaning time is in a range from 10 to 45 minutes.

Clause 63. The method of any of clauses 53-62, wherein an amount of a biological contaminant within the interior chamber is reduced by at least 99.99%.

Clause 64. The method of any of clauses 53-63, wherein an amount of a biological contaminant remaining within the microplate reader is less than that of conventional sterilization techniques.

Clause 65. The method of any of clauses 53-64, further comprising decomposing residual airborne H₂O₂ into water and O₂ gas.

Clause 66. The method of any of clauses 53-65, further comprising washing residual H₂O₂ disinfectant solution on a surface of a component of the microplate instrument.

Cause 67. The method of any of clauses 53-66, wherein the concentration-time product C*T in the interior chamber is in a range from about 100 to about 15,000 ppm/min.

1. A method for sterilizing a microplate instrument comprising an interior chamber, the method comprising:
generating an airborne disinfectant in the interior chamber from a disinfectant solution comprising H₂O₂;
monitoring a cleaning parameter in the interior chamber correlated to an amount of the airborne disinfectant in the interior chamber; and
maintaining the amount of the airborne disinfectant within a predetermined range for a cleaning time sufficient to sterilize the interior chamber according to a SAL-6 requirement, in which an amount of a biological contaminant within the microplate instrument interior chamber is reduced by a log-reduction factor of at least 6 (or 99.99%).

2. The method of claim 1, wherein the airborne disinfectant is a vapor or a suspension.

3. The method of claim 1, wherein generating the airborne agent comprises a cycle, the cycle comprising:
flowing an amount of the disinfectant solution into a vaporization module; and
vaporizing the amount of the disinfectant solution in the vaporization module to ensure that the disinfectant solution is completely vaporized, without creating the possibility of explosive conditions.

4. (canceled)

5. The method of claim 1, wherein the cleaning parameter is a concentration of airborne H₂O₂.

6. The method of claim 1, wherein the cleaning parameter is relative humidity.

7. The method of claim 1, wherein the cleaning parameter is temperature.

8. The method of claim 1, wherein monitoring the cleaning parameter comprises monitoring a parameter within the disinfectant solution.

9. The method of claim 1, wherein:
monitoring the cleaning parameter comprises monitoring relative humidity; and
maintaining the cleaning parameter within the predetermined range comprises maintaining the relative humidity within the interior chamber in a range from 10% to 95%.

10. The method of claim 1, wherein the cleaning time is in a range from 10 to 45 minutes.

11. (canceled)

12. The method of claim 1, wherein an amount of a biological contaminant remaining within the microplate reader is less than that of conventional sterilization techniques.

13. The method of claim 1, further comprising decomposing residual airborne H₂O₂ into water and O₂ gas.

14. The method of claim 1, further comprising washing residual H₂O₂ disinfectant solution on a surface of a component of the microplate instrument.

15. The method of claim 1, wherein the concentration-time product C*T in the interior chamber is in a range from about 100 to about 15,000 ppm/min.

16. The method of claim 1, further comprising calculating the cleaning parameter according to known relationships between the amount of airborne disinfectant generated and the expected decay of the airborne disinfectant.

17. The method of claim 1, further comprising repeatedly vaporizing only a portion of a total amount of the disinfectant solution to be vaporized within a vaporization module.

18. The method of claim 1, wherein the disinfectant solution comprises from 0.1 wt. to 12 wt. % H₂O₂.

19. The method of claim 1, wherein the disinfectant solution comprises from 0.5 wt. to 8 wt. % H₂O₂.

20. The method of claim 1, wherein the disinfectant solution comprises from 1 wt. to 4 wt. % H₂O_{2.}

21. The method of claim 1, further comprising a number of vaporization cycles to preserve the correlation to the airborne disinfectant where decomposition rates of the airborne disinfectant and the airborne solvent differ.
